(19) United States
(12) Patent Application Publication
(10) Pub. No.: US 2023/0210770 A1
(43) Pub. Date: Jul. 6, 2023

(54) TOPICAL OPHTHALMOLOGICAL COMPOSITIONS AND METHODS FOR TREATING ABNORMAL ANGIOGENESIS

(71) Applicant: ADS THERAPEUTICS LLC, Irvine, CA (US)

(72) Inventors: Jinsong NI, Irvine, CA (US); Rong YANG, Irvine, CA (US)

(21) Appl. No.: 17/927,796

(22) PCT Filed: May 11, 2021
(86) PCT No.: PCT/US2021/031780

§ 371 Date: Nov. 25, 2022

Related U.S. Application Data
(60) Provisional application No. 63/032,920, filed on Jun. 1, 2020.

(51) Int. Cl. A61K 9/107; A61K 31/4439; A61K 31/506; A61K 31/496; A61P 27/06; A61K 9/00

(52) U.S. Cl. CPC A61K 9/107; A61K 31/4439; A61K 31/506; A61K 31/496; A61P 27/06; A61K 9/0048

(57) ABSTRACT

A topical ophthalmological composition includes a multikinase inhibitor as an active pharmaceutical ingredient, and perfluorohexyloctane (F6H8) as a liquid vehicle. The multikinase inhibitor inhibits vascular endothelial growth factor receptors (VEGFRs). A method for treating an ophthalmological disorder includes: providing a topical ophthalmological composition containing a multikinase inhibitor at a concentration of about 0.01-10% (w/v); and treating a patient with the topical ophthalmological composition for treating the ophthalmological disorder.

The present application claims priority to U.S. Provisional Application No. 63/032,920, filed on Jun. 1, 2020, which is incorporated by reference for all purposes as if fully set forth herein.

### FIELD OF THE INVENTION

The present invention relates to topical ophthalmological compositions and methods for treating abnormal angiogenesis and fibrosis, more specifically, topical ophthalmological compositions include a multikinase inhibitor as an active pharmaceutical ingredient and perfluorohexyloctane (F6H8 or CF₃(CF₂)₅(CH₂)₇CH₃) as a liquid vehicle, and a method for treating ophthalmological disorders with abnormal angiogenesis.

### BACKGROUND OF THE INVENTION

Multikinase inhibitors are molecules that inhibit multiple kinases. They are often used for cancer treatment. Many of these inhibitors target tyrosine kinase receptors, such as vascular endothelial growth factor receptors (VEGFRs). Inhibitors that target VEGFRs can inhibit new blood vessel formation or abnormal vessel formation under disease conditions. These inhibitors also inhibit, at various potency, the platelet-derived growth factor receptors (PDGFRs) that have roles in blood vessel maintenance. Another family of kinases targeted by these are fibroblast growth factor receptors (FGFRs) that may have roles in fibroblast growth and fibrosis. Inhibitors with such target profiles may be useful for treating diseases with abnormal angiogenesis or vascularity including many ocular diseases. They may also be useful for treating diseases with abnormal fibrosis.

Multikinase inhibitors are well known to be highly hydrophobic small molecules with very low solubility in water or water-based vehicles. Thus, there is a need to develop a formulation so to deliver sufficient concentrations multikinase inhibitors to the target sites to achieve desirable pharmacologic effects.

### SUMMARY OF THE INVENTION

In one embodiment, a topical ophthalmological composition includes: a multikinase inhibitor as an active pharmaceutical ingredient; and perfluorohexyloctane (F6H8) as a liquid vehicle. The multikinase inhibitor inhibits vascular endothelial growth factor receptors (VEGFRs).

In another embodiment, the multikinase inhibitor inhibits VEGFRs and fibroblast growth factor receptors (FGFRs).

In another embodiment, the multikinase inhibitor is selected from the group consisting of axitinib, regorafenib, pazopanib, nintedanib, and a pharmaceutically acceptable salt thereof.

In another embodiment, the multikinase inhibitor has a concentration of about 0.01-0.1% (w/v), about 0.1-1% (w/v), about 1-10% (w/v), about 1.5%-5% (w/v), about 1.5% (w/v), about 2% (w/v), about 2.5% (w/v), about 3% (w/v), about 3.5% (w/v), about 4% (w/v), about 4.5% (w/v), about 5% (w/v), about 6% (w/v), about 7% (w/v), about 8% (w/v), about 9% (w/v), or about 9% (w/v).

In another embodiment, the topical ophthalmological composition is a non-water-based formulation of suspension, solution, or emulsion.

In another embodiment, the liquid vehicle is free of water.

In another embodiment, the topical ophthalmological composition further includes an organic cosolvent selected from the group consisting of ethanol, isopropanol, glycerol, propylene glycol, and polyethylene glycol.

In another embodiment, a topical ophthalmological composition consists of a multikinase inhibitor as an active pharmaceutical ingredient; and perfluorohexyloctane (F6H8) as a liquid vehicle. The multikinase inhibitor inhibits vascular endothelial growth factor receptors (VEGFRs).

In another embodiment, the multikinase inhibitor inhibits VEGFRs and fibroblast growth factor receptors (FGFRs).

In another embodiment, the multikinase inhibitor is selected from the group consisting of axitinib, regorafenib, pazopanib, nintedanib, and a pharmaceutically acceptable salt thereof.

In another embodiment, the multikinase inhibitor has a concentration of about 0.01-0.1% (w/v), about 0.1-1% (w/v), about 1-10% (w/v), about 1.5%-5% (w/v), about 1.5% (w/v), about 2% (w/v), about 2.5% (w/v), about 3% (w/v), about 3.5% (w/v), about 4% (w/v), about 4.5% (w/v), about 5% (w/v), about 6% (w/v), about 7% (w/v), about 8% (w/v), about 9% (w/v), or about 9% (w/v).

In another embodiment, a method for treating an ophthalmological disorder includes: providing a topical ophthalmological composition containing a multikinase inhibitor at a concentration of about 1-10% (w/v); and treating a patient with the topical ophthalmological composition for treating the ophthalmological disorder. The multikinase inhibitor inhibits vascular endothelial growth factor receptors (VEGFRs).

In another embodiment, the multikinase inhibitor inhibits VEGFRs and fibroblast growth factor receptors (FGFRs).

In another embodiment, the multikinase inhibitor is selected from the group consisting of axitinib, regorafenib, pazopanib, nintedanib, and a pharmaceutically acceptable salt thereof.

In another embodiment, the multikinase inhibitor has a concentration of about 0.01-0.1% (w/v), about 0.1-1% (w/v), about 1-10% (w/v), about 1.5%-5% (w/v), about 1.5% (w/v), about 2% (w/v), about 2.5% (w/v), about 3% (w/v), about 3.5% (w/v), about 4% (w/v), about 4.5% (w/v), about 5% (w/v), about 6% (w/v), about 7% (w/v), about 8% (w/v), about 9% (w/v), or about 9% (w/v).

In another embodiment, the topical ophthalmological composition is a non-water-based formulation of suspension, solution, or emulsion, and includes perfluorohexyloctane (F6H8) as a liquid vehicle. Preferably, F6H8 is a sole liquid vehicle, and the liquid vehicle is free of water.

In another embodiment, the ophthalmological disorder is selected from the group consisting of glaucoma surgery failure, minimally invasive glaucoma surgery failure, neovascular glaucoma, meibomian gland dysfunction, dry eye diseases, Sjogren's syndrome, alkali burns, ulceration, graft versus host disease, atopic conjunctivitis, ocular rosacea, cicatricial pemphigoid, stem cell deficiency, Lyell's syndrome, Steven Johnson syndrome, viral, bacterial, fungal, pterygium, pinguecula, cornea transplant infection, cornea parasitic infection, and contact lens induced neovascularization.

In another embodiment, the glaucoma surgery failure results from a classic trabeculectomy, a Trabectome surgery, a gonioscopy-assisted transluminal trabeculotomy, an excimer laser trabeculostomy, and an endoscopic cyclophotocoagulation; the minimally invasive glaucoma surgery failure results from implanting an ocular filtration device.

In another embodiment, the ocular filtration device used in the minimally invasive glaucoma surgery is selected from the group consisting of a subconjunctival stent, a Schlemm's canal stent, and a suprachoroidal stent.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 shows fibroblast density score of Groups 1-4. Group 1: vehicle control; group 2: MMC positive control; group 3: 0.3% nintedanib treatment from—day 7 to day 30; group 4: 0.3% nintedanib from day 1 to day 30.

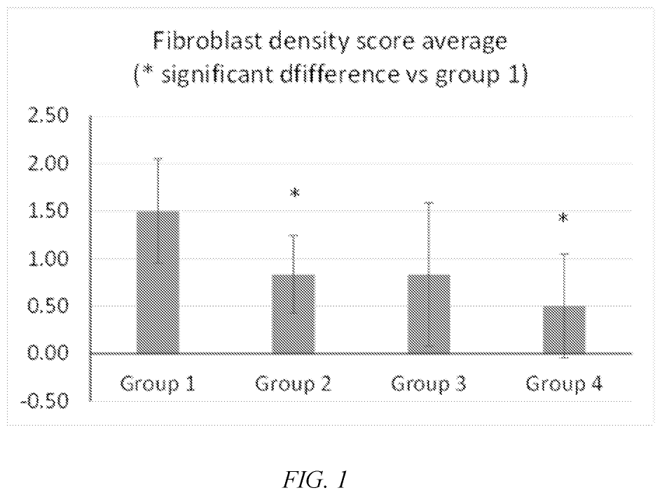

### DETAILED DESCRIPTION OF THE ILLUSTRATED EMBODIMENTS

Reference will now be made in detail to embodiments of the present invention, example of which is illustrated in the accompanying drawings.

Multikinase inhibitor (MM) refers to drug compounds (e.g., a small molecule) that inhibit the activity of two or more kinases, including, for example, intracellular and/or cell surface tyrosine protein kinases. A “small molecule” is understood to refer to a chemical compound having a molecular weight below 2,000 Daltons. It is preferred that these small molecules are organic molecules. In certain embodiments, “small molecule” does not include peptide or nucleic acid molecules.

Exemplary multikinase inhibitors for use in the methods described herein demonstrate certain kinase inhibition profiles, for example, multikinase inhibitors that have a kinase inhibition profile with an in vitro IC₅₀ against VEGFR(1, 2, 3) (IC₅₀<100 nM), PDGFRα (IC₅₀<1000 nM) and FGFR1 (IC₅₀<500 nM). Exemplary multikinase inhibitors for use in the methods described herein include, for example, afatinib, amuvatinib, axitinib, cabozantinib, canertinib, cediranib, ceritinib, crenolanib, crizotinib, dabrafenib, dacomitinib, dasatinib, erlotinib, foretinib, gefitinib, golvatinib, ibrutinib, icotinib, idelalisib, imatinib, lapatinib, lenvatinib, neratinib, nilotinib, nintedanib, palbociclib, pazopanib, ponatinib, quizartinib, regorafenib, ruxolitinib, sorafenib, sunitinib, tandutinib, tivantinib, tivozanib, trametinib, vandetanib, vatalanib, and vemurafenib; preferably, axitinib, nintedanib, pazopanib, cediranib, regorafenib, ponatinib, lenvatinib; and more preferably, axitinib, nintedanib, pazopanib, and regorafenib. The structures and in vitro potencies of the preferred exemplified kinase inhibitors are shown below and in Table 1.

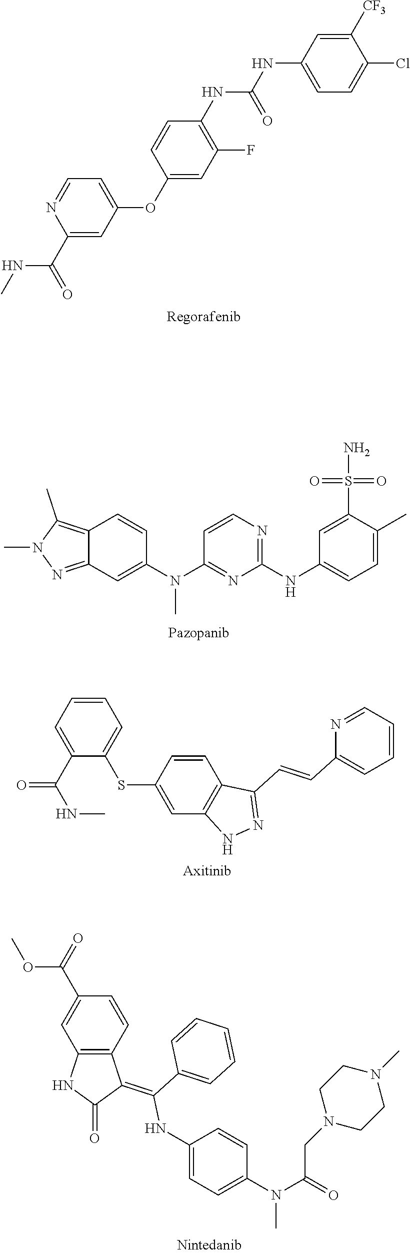

TABLE 1
In vitro potencies of selected kinase inhibitors on key receptors

| | Regorafenib | Pazopanib | Axitinib | Nintedanib |
|---|---|---|---|---|
| VEGFR1 | 10 | 10 | 0.1 | 34 |
| VEGFR2 | 10 | 30 | 0.2 | 21 |
| VEGFR3 | 46 | 47 | 0.2 | 13 |
| PDGFRα | 886 | 71 | 5 | 22 |
| PDGFRβ | NA | 84 | 1.6 | 39 |
| FGFR1 | 26 | 140 | 231 | 69 |
| FGFR2 | 50 | 350 | NA | 37 |
| FGFR3 | NA | 130 | NA | 108 |
| FGFR4 | NA | NA | NA | 610 |

MKIs are commonly used for treating cancers. The mechanism is to inhibit angiogenesis/blood vessel formation at the cancer site and starve the cancer tissues of nutrients supply that regress their growth. This mechanism can be applied to other diseases with abnormal vascularity. Some MKIs, like the examples listed here, can also inhibit FGFRs and have the potential to treat diseases with abnormal fibrosis. The compositions and methods described herein are useful for treating patients with ocular diseases at the front of the eye that involve abnormal angiogenesis/vascularity or fibrosis or both.

For example, the disclosure provides compositions and methods of treatment using one of the listed examples of MKIs, such as regorafenib, for improving the success rate of glaucoma surgery (e.g., glaucoma filtration surgery), by administering to the eye of a subject in need of such treatment. One aspect features a method for adjunctive treatment associated with glaucoma surgery in a subject comprising administering to a subject in need thereof an effective amount of a composition comprising regorafenib or a pharmaceutically acceptable salt thereof. The method improves the success rate of glaucoma surgery. Glaucoma surgery includes, for example, the classic trabeculectomy method, or a method selected from the group consisting of Trabectome surgery, Gonioscopy-assisted transluminal trabeculectomy, Excimer laser trabeculostomy, and Endoscopic cyclophotocoagulation. The glaucoma surgery performed may also be for implantation of an ocular filtration device, wherein the ocular filtration device is an ocular stent. For example, the ocular filtration device may be selected from the group consisting of an iStent, Xen Gel Stent, Hydrus and CyPass microstent.

In another aspect, the disclosed methods reduce scar formation in glaucoma surgery by attenuating abnormal vascularity and fibrosis at the surgical site. In certain aspect, the disclosed methods are performed before operation, in conjunction with operation or after operation, to reduce failure in glaucoma surgery. In some aspects, the amount of regorafenib administered is effective to extend the duration of lower the intraocular pressure (TOP) for at least 10 days, at least 365 days, or at least 3650 days following surgery. In some aspects, the amount of regorafenib administered is effective to prolong bleb survival.

The materials, methods, and examples are illustrative only and not intended to be limiting.

In summary, excess vascularity and fibrosis are key risk factors that lead to excess scarring and failure in glaucoma surgery. The disclosure provides compositions and methods that utilize anti-angiogenic and anti-fibrotic mechanisms to increase the rate of success of glaucoma surgery.

MKIs are well known to be highly hydrophobic small molecules with very low solubility in water or water-based vehicles. For example, axitinib has a solubility of only 0.2 mg/ml in water at neutral pH. As such, they are difficult to formulate into a stable eye drop composition for ocular use. The disclosure provides compositions and methods of treatment using an MKI in a stable formulation composed of perfluorohexyloctane (F6H8) as the liquid vehicle for treatment of ocular diseases with abnormal vascularity and/or fibrosis. Perfluorohexyloctane (F6H8) is an amphiphilic liquid with two mutually immiscible moieties (hydrocarbon segment and perfluorinated segment) bound covalently. Other related analogies used in the compositions of the present inventions may be perfluorobutylpentane (F4H5), perfluorobutylhexane (F4H6), perfluorohexylbutane (F6H4), perfluorohexylhexane (F6H6), perfluorohexyloctane (F6H8), and perfluorohexyldecane (F6H10); preferably, perfluorobutylpentane (F4H5), perfluorohexylhexane (F6H6), and perfluorohexyloctane (F6H8); more preferably, perfluorohexyloctane (F6H8).

The structure of F6H8 is shown below.

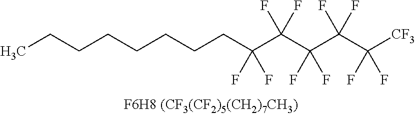 [SMILES: CCCCCCCCC(F)(F)C(F)(F)C(F)(F)C(F)(F)C(F)(F)C(F)(F)F]

In some embodiments, the disclosure is based on the studies described in the examples that show the use of regorafenib formulated in F6H8 is a preferred choice for treating the ocular indications listed in the disclosure. More specifically, the combination is selected for reducing the failure rate of glaucoma surgery.

Example 1 describes the efforts to develop water-based formulation of nintedanib. Nintedanib is highly insoluble in water with the reported solubility less than 0.01 mg/mL, that is 0.001%. Various water-based formulation carriers and their combinations were tested to try to formulate nintedanib to a reasonably high concentration. As a result, a water-based emulsion formulation was discovered to be able to hold 0.3% nintedanib with good long-term stability. Example 2 describes the testing of nintedanib 0.3% in an emulsion formulation in a rabbit glaucoma filtration surgery model. In this example, however, it was discovered that this formulation was not able to deliver enough drug to the surgical site to have the intended effect of preventing bleb failure. At the same time, an efficacy signal was detected to show that fibroblast density was reduced by the formulation. Since fibrosis is a key step in the failure of surgical success, this example indicated that if more nintedanib can be delivered to the surgical site with a more effective vehicle, the intended efficacy may be achieved.

Because the higher concentration of nintedanib might lead to unwanted adverse effects, such as yellow conjunctiva discoloration observed in the clinic due to yellow color of nintedanib molecule, regorafenib, a colorless compound that has similar pharmacologic profile as nintedanib, was selected for further formulation development and in vivo animal model testing. Further experiments were conducted to evaluate various solvents, to determine which solvent would be the best for regorafenib to form a formulation at much higher concentration for topical ocular use. In example 3, among all the solvents tested, F6H8 was a preferred choice as a non-water-based vehicle to formulate regorafenib into a high concentration, for example, about 0.01-0.1% (w/v) or about 0.1-1% (w/v); preferably, about 1-10% (w/v), about 1.5%-5% (w/v), or about 1.5% (w/v); more preferably, about 2% (w/v), about 2.5% (w/v), about 3% (w/v), about 3.5% (w/v), about 4% (w/v), about 4.5% (w/v), about 5% (w/v), about 6% (w/v), about 7% (w/v), about 8% (w/v), about 9% (w/v), or about 9% (w/v). In example 4, a thought-experiment, a 2% regorafenib formulation in F6H8 would improve the success of glaucoma surgery in a dog model. This disclosure of the composition of MKIs formulated with F6H8 is a novel use to deliver sufficient concentrations multikinase inhibitors to the target sites to achieve desirable pharmacologic effects.

### EXAMPLES

### Example 1: Emulsion Formulation for Multikinase Inhibitors

Formulations of nintedanib were investigated according to the following procedure:

1. Tare 1.5 mL Eppendorf tube

2. Add nintedanib and record weight

3. Add solubilizer and record weight

4. Add water pH 5 (except F3 & F4) and record weight

5. Beadbeater mix for 120 seconds

6. Place on rotating mixer for overnight at ambient temperature

7. Filter through 0.2 μm SPIN-X centrifuge filter

8. Measure pH of filtrate

9. Assay filtrate using CBT-001 standard solution

The vastly different results were obtained for the investigated solubilizers despite many of the investigated solubilizers having similar structural properties (see, e.g., Tables 2 and 3). It was discovered that solubilizers such as castor oil and polysorbate 80 were found to have high solubilizing performance for nintedanib.

TABLE 2
Compositions screened (% wt)

| % WT | F-1 | F-2 | F-3 | F-4 | F-5 | F-6 | F-7 | F-8 | F-9 | F-10 | F-11 | F-12 |
|---|---|---|---|---|---|---|---|---|---|---|---|---|
| Nintedanib | 0.5 | 0.5 | 0.5 | 0.5 | 0.5 | 0.5 | 0.5 | 0.5 | 0.5 | 0.5 | 0.5 | 0.5 |
| Benzyl alcohol | 1 | | | | | | | | | | | |
| Ethanol | | 1 | | | | | | | | | | |
| Castor oil | | | 99.5 | | | | | | | | | |
| Mineral oil | | | | 99.5 | | | | | | | | |
| BZK | | | | | 1 | | | | | | | |
| Polysorbate 20 | | | | | | 1 | | | | | | |
| Polsorbate 80 | | | | | | | 1 | | | | | |
| Poloxamer 188 | | | | | | | | 1 | | | | |
| Poloxamer 407 | | | | | | | | | 1 | | | |
| PEG 400 | | | | | | | | | | 5 | | |
| PEG 8000 | | | | | | | | | | | 2 | |
| Propylene | | | | | | | | | | | | 1 |
| WFI | 98.5 | 98.5 | | | 98.5 | 98.5 | 98.5 | 98.5 | 98.5 | 94.5 | 97.5 | 98.5 |

TABLE 3
Solubility of nintedanib in various solubilizers

| Solubilizer | Nintedanib solubility (mg/g) | pH |
|---|---|---|
| BZA | 0.01 | 5.6 |
| EtOH | 0.01 | 6.1 |
| Castor oil | 0.37 | — |
| Mineral oil | 0.00 | — |
| BZK | 0.20 | 5.6 |
| polysorbate 20 | 0.06 | 6.6 |
| polysorbate 80 | 0.25 | 6.7 |
| PLXMR 188 | 0.01 | 6.5 |
| PLXMR 407 | 0.01 | 6.3 |
| PEG 400 | 0.17 | 5.6 |
| PEG 8000 | 0.01 | 6.0 |
| PG | 0.01 | 6.0 |

Various water-based emulsion systems were identified for investigation based on the solubilizer results. It was found that water-based emulsion systems combining castor oil, polysorbate 80 and polyoxyl-35 castor oil can suitably solubilize nintedanib. As shown in Table 4, one of the emulsion systems can dissolve nintedanib to about 3-5 mg/ml, that is 0.3%-0.5% of ophthalmic emulsion concentration.

TABLE 4
Solubility of nintedanib in a representative emulsion system.

| ID | F70 lot 1 | F70 lot 2 |
|---|---|---|
| Castor Oil | 1 | 1 |
| Polysorbate 80 | 1 | 1 |
| Polyoxyl-35 castor oil | 2 | 2 |
| Water | 95 | 95 |
| Nintedanib solubility | 5 | 3.4 |

### Example 2: Rabbit Glaucoma Filtration Surgery Model Study of Nintedanib

In this example, a nintedanib 0.3% emulsion was tested in a rabbit glaucoma filtration surgery model. Nintedanib is one of the four MM examples that can inhibit angiogenesis and fibrosis. The study showed a positive efficacy signal on fibroblast density but not bleb survival, suggesting higher level of drug is needed to be potentially useful in real glaucoma surgery.

Methods

Model set up: The glaucoma filtration surgery model was established as previously described (Cordeiro et al. 1997; Zhong et al. 2011).

Negative control: vehicle BID dosing from minus Day 7-Day 30 (group 1).

Positive control: mitomycin C (MMC) for 5 min during surgery (group 2).

Testing article: 0.3% CBT-001 (nintedanib), BID dosing from minus Day 7-Day 30 (group 3) and from Day 1-Day 30 (group 4).

Measurements: TOP, bleb score, histology for fibroblast density, fibrosis staining and inflammation level.

Time points: Days 2, 4, 7, 10, 14, 21 and 30 post surgery.

Animals: 24 female New Zealand White rabbits (6/group).

The study design is shown in Table 5 below.

TABLE 5
Study design

| | | Filtration | Test Article | | |
|---|---|---|---|---|---|
| | # of | Surgery | Treatment | End Point | |
| Group | Animals | (Day 1) | (BID) | Parameters | Termination |
| 1 | 6 | OS: | OU: Vehicle | OU: | Day 30 |
| | | Filtration | Control lot: | OEs and IOPs | |
| | | surgery | 309-2-20-130 | at baseline and | |
| | | OD: no | (7 days pre- | at D2, D4, D7, | |
| | | surgery | surgery and 30 | D10, D14, | |
| | | | days after | D21, and D30 | |
| | | | surgery) | OS: | |
| 2 | 6 | OS: | OS: Positive | Digital | |
| | | Filtration | control (MMC | photography, | |
| | | surgery | lot: 20150601 | survival, size | |
| | | OD: no | 0.4 mg/mL | and morphology of | |
| | | surgery | soaked 8 × 10 | blebs at D2, D4, | |
| | | | mm sponge for | D7, D10, D14, | |
| | | | 5 min as | D21, and D30 | |
| | | | described by | H&E staining | |
| | | | Cordeiro et al) | (terminal) | |
| 3 | 6 | OS: | OU: 0.3% | Masson | |
| | | Filtration | CBT-001 (7 | Trichrome | |
| | | surgery | days pre- | staining | |
| | | OD: no | surgery and 30 | (terminal) | |
| | | surgery | days after | Picrosirius red | |
| | | | surgery, BID) | staining | |
| 4 | 6 | OS: | OU: 0.3% | (terminal) | |
| | | Filtration | CBT-001 (30 | OD: (Group 3 | |
| | | surgery | days after | and 4 only PK | |
| | | OD: no | surgery, BID) | analysis) | |
| | | surgery | | (terminal) | |

OU = both eyes;
OD = right eye;
OS = left eye;
IOP = Intraocular pressure;
OE = ocular exam;
H&E = Hematoxylin and eosin stain;
PK = pharmacokinetics

Results and Conclusion

Among all the measurements, only the fibroblast density showed a signal of efficacy. The results are shown in FIG. 3. We observed a significant reduction of fibroblast density in group 4 (treated with nintedanib for 30 days A trend of reduction not statistically significant is also seen in group 3 (treated with nintednib from −Day 7 to Day 30). The main efficacy endpoint, bleb survival, didn't show any difference from the vehicle control in the two treatment groups.

From the results, we concluded that the nintedanib 0.3% emulsion was not effective in improving bleb survival. However, a signal of efficacy was detected in fibroblast density. We further concluded that the current 0.3% formulation did not deliver enough drug to the surgical site but the mechanism of multikinase inhibitor reducing failure of glaucoma surgery is valid. The results indicated that a MM with target pharmacologic profiles like nintedanib will have efficacy if sufficient amount can be delivered to the surgical site.

### Example 3: Formulating Regorafenib into a Non-Water Based Eyedrop with 2% Concentration

In this example, experiments were carried out to formulate regorafenib to a high concentration of, e.g. 2%, for topical ocular use. F6H8 was identified as a non-water-based vehicle best suited for the purpose.

400 mg of micronized regorafenib monohydrate was suspended in 20 ml of perfluorohexyloctane (F6H8). The Suspension was homogenized by stirring at room temperature for 15 minutes. Measured regorafenib concentration ranged between 95.4-99.1% of theoretical concentration. The observed fluctuation was most likely due to inhomogeneity of the sample after manual shaking of the suspension. No unidentified degradation product was observed in the chromatograms using the HPLC method described below.

HPLC method for the determination of regorafenib concentration was the following: Samples were prepared by dilution of drawn formulation aliquots with water: acetonitrile (25/75) to a final regorafenib concentration of 100 ug/ml. 100 of each sample were injected into an Agilent 1100 HPLC system (Agilent, Waldbronn, Germany), and samples were run on a heated (40° C.) Symmetry C18 column (150×4.6 mm-3.5 um particle size, Waters, Eschborn, Germany) applying a flow rate of 1 ml/min. The mobile phase consisted of a mixture of potassium phosphate buffer pH 2.4 (A) and acetonitrile/ethanol (6/4) (B). The following gradient was applied: minute 0: A, 60%/B, 40%: minute 12: A, 20%/B, 80%: minute 16: A, 20%/B, 80%: minute 16.5: A, 60%/B, 40%: minute 20: A, 60%/B, 40%. Regorafenib was quantified using a DAD detector at a wavelength of 265 nm. The regorafenib peak appeared at 12.5 minutes.

### Example 4: Dog Glaucoma Filtration Surgery Model Study of Regorafenib

In this thought-experiment example, a 2% regorafenib formulation prepared in perfluorohexyloctane (F6H8) vehicle is tested in a dog model of glaucoma filtration surgery. Regorafenib another MM examples in the disclosure that can inhibit angiogenesis and fibrosis. It's in vitro potencies on VEGFR and FGFR are about two-fold more than that of nintedanib. The study shows that the formulation can improve the success rate of glaucoma surgery in the dog model and suggest that it will be potentially useful in human glaucoma surgery to reduce failure rate.

Methods

Model set up: The glaucoma filtration surgery model was established as previously described (Kojima et al. 2015). After surgery, ofloxacin ointment will be applied to the eye.

Testing article: 2% regorafenib in perfluorohexyloctane, BID for 4 weeks after surgery.

Control: perfluorohexyloctane, BID for 4 weeks after surgery.

Measurements: IOP, bleb score, collagen level.

Time points: Day14 and 28 post surgery.

The study design is shown in Table 6 below.

TABLE 6
Study design

| | | Filtration | Test Article | | |
|---|---|---|---|---|---|
| | # of | Surgery | Treatment | End Point | |
| Group | Animals | (Day 1) | (BID) | Parameters | Termination |
| 1 | 6 | OS: | OU: Vehicle | OU: | Day 28 |
| | | Filtration | Control lot: 309- | OEs and IOPs | |
| | | surgery | 2-20-130 | at baseline and | |
| | | OD: no | (Immediately | at D14, D28 | |
| | | surgery | after surgery, | OS: | |
| | | | BID for 28 days) | Digital | |
| 2 | 6 | OS: | OU: 1% CBT- | photography, | |
| | | Filtration | 001 lot: 309-2- | survival, size | |
| | | surgery | 20-118 | and morphology of | |
| | | OD: no | (Immediately | blebs at D14, | |
| | | surgery | after surgery, | D28 H&E staining | |
| | | | BID for 28 days) | (terminal) | |
| 3 | 6 | OS: | OU: 2% CBT- | Masson | |
| | | Filtration | 001 lot: 309-2- | Trichrome | |
| | | surgery | 20-118 | staining | |
| | | OD: no | (Immediately | (terminal) | |
| | | surgery | after surgery, | | |
| | | | BID for 28 days) | | |

OU = both eyes;
OD = right eye;
OS = left eye;
IOP = Intraocular pressure;
OE = ocular exam;
H&E = Hematoxylin and eosin stain;
PK = pharmacokinetics

Results and Conclusion

As shown in Table 7, the regorafenib treatment group would show significant differences from the vehicle group. Lower IOP is observed on Day 14 and 28 versus no significant change in vehicle. The bleb score is higher in the regorafenib group than vehicle group and the collagen level is lower in regorafenib group.

TABLE 7
Example 3 results on IOP, bleb score and collagen density

| | | |
|---|---|---|
| Day 1 (baseline) | Day 14 | Day 28 |

Vehicle

| | | | |
|---|---|---|---|
| IOP (mmHg) | 16.1 | 11.6 | 13.2 |
| Bleb score | NA | 2.3 | 1.2 |
| Collagen density (%) | NA | NA | 85.3 |

Drug

| | | | |
|---|---|---|---|
| IOP (mmHg) | 16.0 | 8.2^{a} | 11.1^{a} |
| Bleb score | NA | 3.5^{b} | 2.3^{b} |
| Collagen density (%) | NA | NA | 71.7 |

^{a}significantly different from baseline.
^{b}Significantly different from vehicle group.

The results indicate that regorafenib at 2% in perfluorohexyloctane (F6H8) formulation can improve success of glaucoma filtration surgery in the dog model. This or a similar formulation of regorafenib will have the potential for use in human glaucoma surgery to reduce failure rate over time. It can also be used for treating other ocular diseases in the front of the eye that involve abnormal vascularity and/or fibrosis.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

1. A topical ophthalmological composition comprising:
a multikinase inhibitor as an active pharmaceutical ingredient; and
perfluorohexyloctane (F6H8) as a liquid vehicle,
wherein the multikinase inhibitor inhibits vascular endothelial growth factor receptors (VEGFRs).

2. The topical ophthalmological composition according to claim 1, wherein the multikinase inhibitor inhibits VEGFRs and fibroblast growth factor receptors (FGFRs).

3. The topical ophthalmological composition according to claim 1, wherein the multikinase inhibitor is selected from the group consisting of axitinib, regorafenib, pazopanib, nintedanib, and a pharmaceutically acceptable salt thereof.

4. The topical ophthalmological composition according to claim 1, wherein the multikinase inhibitor has a concentration of about 0.01-0.1% (w/v), about 0.1-1% (w/v), about 1-10% (w/v), about 1.5%-5% (w/v), about 1.5% (w/v), about 2% (w/v), about 2.5% (w/v), about 3% (w/v), about 3.5% (w/v), about 4% (w/v), about 4.5% (w/v), about 5% (w/v), about 6% (w/v), about 7% (w/v), about 8% (w/v), about 9% (w/v), or about 9% (w/v).

5. The topical ophthalmological composition according to claim 1, wherein the topical ophthalmological composition is a non-water-based formulation of suspension, solution, or emulsion.

6. The topical ophthalmological composition according to claim 1, wherein perfluorohexyloctane (F6H8) is a liquid vehicle of the topical ophthalmological composition.

7. The topical ophthalmological composition according to claim 1, further comprising an organic cosolvent selected from the group consisting of ethanol, isopropanol, glycerol, propylene glycol, and polyethylene glycol.

8. A topical ophthalmological composition consisting of:
a multikinase inhibitor as an active pharmaceutical ingredient; and
perfluorohexyloctane (F6H8) as a liquid vehicle,
wherein the multikinase inhibitor inhibits vascular endothelial growth factor receptors (VEGFRs).

9. The topical ophthalmological composition according to claim 8, wherein the multikinase inhibitor inhibits VEGFRs and fibroblast growth factor receptors (FGFRs).

10. The topical ophthalmological composition according to claim 1, wherein the multikinase inhibitor is selected from the group consisting of axitinib, regorafenib, pazopanib, nintedanib, and a pharmaceutically acceptable salt thereof.

11. The topical ophthalmological composition according to claim 1, wherein the multikinase inhibitor has a concentration of about 0.01-0.1% (w/v), about 0.1-1% (w/v), about 1-10% (w/v), about 1.5%-5% (w/v), about 1.5% (w/v), about 2% (w/v), about 2.5% (w/v), about 3% (w/v), about 3.5% (w/v), about 4% (w/v), about 4.5% (w/v), about 5% (w/v), about 6% (w/v), about 7% (w/v), about 8% (w/v), about 9% (w/v), or about 9% (w/v).

12. A method for treating an ophthalmological disorder comprising:
providing a topical ophthalmological composition containing a multikinase inhibitor at a concentration of about 0.01-10% (w/v); and
treating a patient with the topical ophthalmological composition for treating the ophthalmological disorder,
wherein the multikinase inhibitor inhibits vascular endothelial growth factor receptors (VEGFRs).

13. The method according to claim 12, wherein the multikinase inhibitor inhibits VEGFRs and fibroblast growth factor receptors (FGFRs).

14. The method according to claim 1, wherein the multikinase inhibitor is selected from the group consisting of axitinib, regorafenib, pazopanib, nintedanib, and a pharmaceutically acceptable salt thereof.

15. The method according to claim 1, wherein the multikinase inhibitor has a concentration of about 0.01-0.1% (w/v), about 0.1-1% (w/v), about 1-10% (w/v), about 1.5%-5% (w/v), about 1.5% (w/v), about 2% (w/v), about 2.5% (w/v), about 3% (w/v), about 3.5% (w/v), about 4% (w/v), about 4.5% (w/v), about 5% (w/v), about 6% (w/v), about 7% (w/v), about 8% (w/v), about 9% (w/v), or about 9% (w/v).

16. The method according to claim 1, wherein the topical ophthalmological composition is a non-water-based formulation of suspension, solution, or emulsion; comprises perfluorohexyloctane (F6H8) as a liquid vehicle; and the liquid vehicle is free of water.

17. The method according to claim 1, wherein the ophthalmological disorder is selected from the group consisting of glaucoma surgery failure, minimally invasive glaucoma surgery failure, neovascular glaucoma, meibomian gland dysfunction, dry eye diseases, Sjogren's syndrome, alkali burns, ulceration, graft versus host disease, atopic conjunctivitis, ocular rosacea, cicatricial pemphigoid, stem cell deficiency, Lyell's syndrome, Steven Johnson syndrome, viral, bacterial, fungal, pterygium, pinguecula, cornea transplant infection, cornea parasitic infection, and contact lens induced neovascularization.

18. The method according to claim 17, wherein the glaucoma surgery failure results from a classic trabeculectomy, a Trabectome surgery, a gonioscopy-assisted transluminal trabeculotomy, an excimer laser trabeculostomy, and an endoscopic cyclophotocoagulation; the minimally invasive glaucoma surgery failure results from implanting an ocular filtration device.

19. The method according to claim 18, wherein the ocular filtration device is selected from the group consisting of a subconjunctival stent, a Schlemm's canal stent, and a suprachoroidal stent.
